(19) United States
(12) Patent Application Publication
(10) Pub. No.: US 2026/0224482 A1
(43) Pub. Date: Aug. 6, 2026

(54) USE OF OXIDATIVE PRECURSORS IN POULTRY FARMING

(71) Applicant: AGA NANOTECH LTD, Hertfordshire (GB)

(72) Inventor: Adrian Fellows, Hertfordshire (GB)

(21) Appl. No.: 19/153,128

(22) PCT Filed: Feb. 2, 2023
(86) PCT No.: PCT/GB2023/050233

§ 371 Date: Aug. 1, 2025

(51) Int. Cl. A61K 9/08; A61K 9/00; A61K 31/19

(52) U.S. Cl. CPC A61K 9/08; A61K 9/0095; A61K 31/19

(57) ABSTRACT

A method for preparing an aqueous solution or dispersion including peracetic acid, including the steps of adding a peroxygen donor and an acetyl donor to an aqueous solution or dispersion to prepare an aqueous solution or dispersion including the peroxygen donor and the acetyl donor, which generate peracetic acid and hydrogen peroxide over the course of time, and adding an organic acid to the aqueous solution or dispersion 10 min or later, such as 15 min or later after the preparation of the aqueous solution or dispersion including the peroxygen donor and the acetyl donor to buffer the pH of the aqueous solution or dispersion. The aqueous solution or dispersion obtained according to the method is suitable for use in an antimicrobial intervention to protect animals, preferably poultry, in particular chicken.

### CROSS REFERENCE TO RELATED PATENT APPLICATIONS

This is a U.S. national phase patent application of PCT/EP2023/050233 filed Feb. 2, 2023, the entire contents of which are incorporated herein by reference for all purposes.

### TECHNICAL FIELD

The present invention relates to a method for preparing an aqueous solution or dispersion comprising peracetic acid, the aqueous solution or dispersion prepared according to the method and its use in an antimicrobial intervention to protect animals.

### BACKGROUND

Antibiotic resistance is the major global problem facing healthcare. Nowhere is this more important than in animal health and husbandry, where misuse and overuse of antibiotics not only as anti-infectives, but also as growth promoters has created widespread reservoirs of resistant infection capable in some cases of transferring to the human population.

A number of countries have now severely limited the use of antibiotics in animal rearing, for example their use in animal feed was banned in China from summer of 2020.

Replacing antibiotics in animal husbandry poses a different range of problems to that in a human medicine.

Here the primary objective is reduction or elimination of pathogens in the gastro intestinal tract, or otherwise modifying the gut microbiome to enhance the health of the animal and as collateral benefit deliver improvements in population survival rates, body weight gain, and feed intake efficiency. At the same time, the organoleptic properties and safety of the reared food product must not be compromised.

Compared to treating human disease, the economic considerations of treating billions of feed animals, for example just poultry are of a different type and magnitude. The treatment can only cost pence per bird life cycle as compared to maybe thousands of pounds per patient in human medicine. This is especially important in applying the treatment to animals in low and middle income countries and across a range of agricultural enterprises from the small holding to the largest industrialized units.

Accordingly, there exists a need to develop novel products which allow the replacement of antibiotics.

Highly oxidative molecules exert a powerful biocidal effect and microorganisms by inflicting massive and widely disseminated damage to the bacterial cell owing to the high energy transfer capability of these molecules. Therefore, resistance to these chemicals is rare. Examples of such molecules would be ozone, hypochlorous acid, sodium hypochlorite or hypobromite, chlorine dioxide, peracids such as peracetic acid and hydrogen peroxide.

Hydrogen peroxide is widely used in endodontics, in the treatment of periodontitis and as an antiseptic for wounds and mucous membranes. Dilute solutions of hypochlorous acid have been advocated for topical disinfection and peracetic acid has been widely used in dairy hygiene as a pre- and post-milking teat disinfectant.

In animal husbandry, there are two basic methods for delivery of long term treatments which may take place over a period of days or even longer. These are in either feed or water.

In water, it is necessary to be able to apply the treatment in both experimental and large scale practical application in a manner that ensures a controlled and quantifiable dose over time.

In experimental conditions or in a small holding this may be a relatively simple matter of direct provision of a measured dose of particles or solution in a small volume of water to a small number of birds, such as maybe 4 to 20 birds. In a large farm unit, it may be necessary to achieve a similar result in a system of hundreds of meters of pipes and multiple drinking stations.

In the prior art there are recorded attempts to achieve similar objectives to the present invention. U.S. Pat. No. 6,342,528 and WO2012/014016A1, US2006/0088498 and US2010/0196503 all cite methods of applying similar chemistries to that used in the present invention to control or prevent microbial growth by administering water treated with a biocide or biocidal composition.

All cite the use of percarboxylic acids in this context. Peracetic acid being a widely used biocide known to those skilled in the art for having excellent antimicrobial properties combined with low toxicity and leaving residual breakdown products benign to both living organisms and the environment.

However, the methods of producing the peracid described in these documents cited above have been found to be unreliable for a treatment of feed animals, particularly poultry and pigs, that is both effective and has no deleterious effects on the treated animals and which requires very precise control of the concentration, pH and residual chemistry.

### SUMMARY

The object of the present invention was to overcome the problems of the prior art cited described above. In particular, the object of the present invention was to provide an aqueous solution or dispersion suitable as animal drink or animal feed and a method for its preparation, which achieves a suitable peracetic acid (PAA) concentration in the aqueous solution or dispersion, e.g. 30-70 ppm, preferably 40-60 ppm, in a relatively constant manner over the time. The suitable PAA concentration should be reached after a relatively short time.

The inventors have now surprisingly found that the object can be achieved by the following method. Administering a two component system to an aqueous solution or dispersion, wherein the first component is a mixture of an acetyl donor and a peroxygen donor, preferably in combination with a chelating agent. The second component is an organic acid to buffer the pH value (pH modifying agent) which is only mixed with the first mixture after a defined time period selected to optimize the release of peracid in the water supply. The levels and precise ratios of the chemicals in both components may be adjusted to provide optimum dosing taking account of the metal ion content and pH of the particular farm water supply.

Accordingly, the present invention is directed to a method for preparing an aqueous solution or dispersion comprising peracetic acid, comprising the steps of: adding a peroxygen donor and an acetyl donor to an aqueous solution or dispersion to prepare an aqueous solution or dispersion comprising the peroxygen donor and the acetyl donor, which generate peracetic acid and hydrogen peroxide over the course of time, and adding an organic acid to the aqueous solution or dispersion 10 min or later, such as 15 min or later after the preparation of the aqueous solution or dispersion comprising the peroxygen donor and the acetyl donor to buffer the pH of the aqueous solution or dispersion.

### DESCRIPTION OF THE DRAWINGS

FIG. 1 is a graphical representation of PAA Generation—adding SP, TAED, EDTA and Citric acid all together at once showing PAA concentration versus time according to an embodiment of the invention.

FIG. 2 is a graphical representation of PAA Generation—adding Citric Acid 5 Minutes after SP, TAED and EDTA showing PAA concentration versus time according to an embodiment of the invention.

FIG. 3 is a graphical representation of PAA Generation—adding Citric Acid 10 Minutes after SP, TAED and EDTA showing PAA concentration versus time according to an embodiment of the invention.

FIG. 4 is a graphical representation of PAA Generation—adding Citric Acid 15 Minutes after SP, TAED and EDTA showing PAA concentration versus time according to an embodiment of the invention.

FIG. 5 is a graphical representation of PAA Generation—adding Citric Acid 30 Minutes after SP, TAED and EDTA showing PAA concentration versus time according to an embodiment of the invention.

FIG. 6 is a graphical representation showing body weight gain (BWG) from D7 to D14 versus log crop bacterial number (BN) according to an embodiment of the invention

### DESCRIPTION OF AN EMBODIMENT

The ensuing description provides some embodiment(s) of the invention, and is not intended to limit the scope, applicability or configuration of the invention or inventions. Various changes may be made in the function and arrangement of elements without departing from the scope of the invention as set forth herein. Some embodiments maybe practiced without all the specific details. For example, circuits may be shown in block diagrams in order not to obscure the embodiments in unnecessary detail. In other instances, well-known circuits, processes, algorithms, structures and techniques may be shown without unnecessary detail in order to avoid obscuring the embodiments.

Reference will be made in detail to embodiments, examples of which are illustrated in the accompanying figures. In the following detailed description, numerous specific details are set forth in order to provide a thorough understanding of the subject matter herein. However, it will be apparent to one of ordinary skill in the art that the subject matter may be practiced without these specific details. In other instances, well known methods, procedures, components, and systems have not been described in detail so as not to unnecessarily obscure features of the embodiments. In the following description, it should be understood that features of one embodiment may be used in combination with features from another embodiment where the features of the different embodiment are not incompatible.

It will also be understood that, although the terms first, second, etc. may be used herein to describe various elements, these elements should not be limited by these terms. These terms are only used to distinguish one element from another. For example, a first object or step could be termed a second object or step, and, similarly, a second object or step could be termed a first object or step. The first object or step, and the second object or step, are both objects or steps, respectively, but they are not to be considered the same object or step.

The terminology used in the description of the disclosure herein is for the purpose of describing particular embodiments only and is not intended to be limiting of the subject matter. As used in this description and the appended claims, the singular forms “a”, “an” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “includes,” “including,” “comprises,” and/or “comprising,” when used in this specification, specify the presence of stated features, integers, steps, operations, elements, and/or components, but do not preclude the presence or addition of one or more other features, integers, steps, operations, elements, components, and/or groups thereof.

In the following, the invention is described in detail.

The unit ppm as used herein refers to ppm by weight unless otherwise stated. An aqueous solution or dispersion as used herein also encompasses an aqueous emulsion.

According to the method of the invention for preparing an aqueous solution or dispersion comprising peracetic acid, the first step comprises:
adding a peroxygen donor and an acetyl donor to an aqueous solution or dispersion to prepare an aqueous solution or dispersion comprising the peroxygen donor and the acetyl donor, which generate peracetic acid and hydrogen peroxide in the course of time.

The rate of the reaction is not very fast so that the generation of peracetic acid and hydrogen peroxide proceeds over a certain time span.

The aqueous solution or dispersion to which the peroxygen donor and the acetyl donor is added is preferably water, more preferably drinking water such as tap water, council water, domestic water. The term drinking water here refers to drinking water suitable for animals, in particular for poultry, such as chicken, ducks and/or turkeys, in particular chicken.

The aqueous solution or dispersion, preferably water, more preferably drinking water, may further comprise one or more additives such as dyes, flavors, taste masking agents, emulsifiers, enhancers, chelating agents, essential oils, herbal extracts, organic fatty acids, nutritional supplements, items of traditional Chinese medicine and/or preservatives. The one or more additives can be added to the aqueous solution or dispersion before, during and/or after, preferably before, the addition of the peroxygen donor and an acetyl donor. A preferred additive is a chelating agent, which is discussed below in more detail.

The aqueous solution or dispersion, preferably water, in particular drinking water, to which the peroxygen donor and the acetyl donor is added has preferably a pH value in the range of 5.0 to 9.5, preferably 6.0 to 9.0 or 6.5 to 9.0, more preferably 7.0 to 8.5.

The peroxygen donor is a compound that is capable of forming hydrogen peroxide in the aqueous solution or dispersion. In general, the acetyl donor is a compound that is capable to react with the hydrogen peroxide generated to produce a mixture of peracetic acid and hydrogen peroxide.

In general, the peroxygen donor and the acetyl donor can be added independently from each other as a fluid, e.g. as a solution in a solvent such as water, or more preferably as a solid such as a powder. The peroxygen donor and the acetyl donor can be added to the aqueous solution or dispersion separately or as a mixture. It is preferred that the peroxygen donor and the acetyl donor are added simultaneously or nearly simultaneously, e.g. within less than 2 min, more preferably within 1 min, wherein a simultaneous addition is preferred.

In a preferred embodiment, the peroxygen donor and the acetyl donor are added as a solid mixture of the peroxygen donor and the acetyl donor to the aqueous solution or dispersion.

In a preferred embodiment, the aqueous solution or dispersion, preferably water, in particular drinking water, comprising the peroxygen donor and the acetyl donor further comprise a chelating agent, i.e. a chelating agent is also added to the aqueous solution or dispersion. The chelating agent can be added before, during or after the addition of the acetyl donor and the acetyl donor, preferably before or during the addition of the acetyl donor and the acetyl donor. The chelating agent can be added in solid form such as a powder or as a fluid, such as a solution of the chelating agent in a solvent such as water, wherein it is preferably added in solid form.

If a chelating agent is also added to the aqueous solution or dispersion, it is a preferred embodiment that the peroxygen donor, the acetyl donor and the chelating agent are added as a solid mixture of the three components to the aqueous solution or dispersion.

The inventors have found that metal ions which may be present in an aqueous solution or dispersion, such as water, in particular drinking water, can interfere with the reaction of the peroxygen donor and the acetyl donor to produce a mixture of peracetic acid and hydrogen peroxide. Chelating agents can mask metal ions present in the aqueous solution or dispersion such as e.g. sodium, potassium, magnesium, calcium and/or iron ions. The addition of a chelating agent is beneficial if metal ions are present in the aqueous solution or dispersion, which significantly affects the reaction the peroxygen donor and the acetyl donor.

In this regard, the addition of chelating agent serves to mask interfering metal ions in the aqueous solution or dispersion, which improves the desired reaction between the peroxygen donor and the acetyl donor. On the other hand, chelating agent usually have acidic characteristics so that the pH of the aqueous solution or dispersion can be increased, if the amount of the chelating agent added is too high. In addition, an increased pH value interferes with the reaction of the peroxygen donor and the acetyl donor to produce a mixture of peracetic acid and hydrogen peroxide which is not desired. Therefore, an amount of chelating agent added to the aqueous solution or dispersion should be rather low.

If the chelating agent is added, the amount of the chelating agent may be, for instance, not more than 0,050 wt. %, more preferably 0,002 to 0,040 wt. %, more preferably 0,005 to 0,020 wt. %, based on the weight of the aqueous solution or dispersion.

With respect to the peroxygen donor, any compound known to be suitable as peroxygen donor or a mixture thereof can be used. The peroxygen donor can be selected e.g. from sodium perborate, ammonium perborate, sodium percarbonate, potassium percarbonate, ammonium percarbonate, sodium perphosphate, ammonium persulphate, urea peroxide, peresters, superoxides, dioxygenyl, ozones, hydrogen peroxide, lithium peroxide, barium peroxide, di-tert-butyl peroxide, ammonium peroxydisulphate and potassium peroxymonosulphate or any mixture thereof, wherein sodium percarbonate, potassium percarbonate, ammonium percarbonate, sodium perborate, ammonium perborate, ammonium persulphate, urea peroxide or any mixture thereof are particularly preferred. The most preferable peroxygen donor is sodium percarbonate (SP).

With respect to the acetyl donor, any compound known to be suitable as acetyl donor or a mixture thereof can be used. For instance, the acetyl donor can be selected from tetraacetylethylenediamine (TAED), methyl cellulose encapsulated TAED, acetyl salicylic acid, diacetyl dioxohexahydratriazine (DADHT), tetraacetyl glycoluril, acetyl urea, di-acetyl urea, tri-acetyl urea, pentaacetyl glucose (PAG), tetraacetyl glycoluril (TAGU), acetyl phosphate, acetyl imidazole, acetyl COA, acetic anhydride, compounds containing a hemiacetal group, acetic acid, diacetylmorphine, pyruvate, acetyl chloride, acetyl-caprolactam and N′N′-diacetyl-N′N′-dimethyl urea or any mixture thereof.

The use of tetraacetylethylenediamine (TAED) as the acetyl donor is especially preferred. It may also be the case in execution of this invention that water may be pretreated with hydrogen peroxide, in which case a reduced level of acetyl donor will be required.

With respect to the chelating agent, any compound known to be suitable for masking (chelating) metal ions can be used. Examples for the chelating agent are ethylenediaminetetraacetic acid (EDTA) and 1-hydroxy ethylidene-1,1-diphosphonic acid (HEDP).

In a preferred embodiment of the inventive method, the peroxygen donor and the acetyl donor are added to the aqueous solution or dispersion in such amounts that the weight ratio of the peroxygen donor to the acetyl donor is from 4:1 to 1:0.5, preferably 3:1 to 1:1, more preferably 2:1 to 1:1.

It is preferred that the peroxygen donor is added, based on the weight of the aqueous solution or dispersion, in an amount in the range of 0.0135 to 0.0315 wt. %, 0.0155 to 0.0300 wt. %, more preferably 0.0200 to 0.0250 wt. %.

It is preferred that the acetyl donor is added, based on the weight of the aqueous solution or dispersion, in an amount in the range of 0.0090 to 0.0210 wt. %, preferably 0.0100 to 0.0200 wt. %, more preferably 0.0135 to 0.0165 wt. %.

It is usually appropriate to stir the aqueous solution or dispersion to which the peroxygen donor and the acetyl donor has been added, preferably the aqueous solution or dispersion is stirred at least until the addition of the organic acid.

In a subsequent step of the inventive method, an organic acid is added to the aqueous solution or dispersion 10 min or later, such as 15 min or later after the preparation of the aqueous solution or dispersion comprising the peroxygen donor and the acetyl donor to buffer the pH of the aqueous solution or dispersion. That is, the starting point is when both the peroxygen donor and the acetyl donor are included in the aqueous solution or dispersion. The addition of the organic acid takes place at the earliest 10 min after this starting point, such as at the earliest 15 min after this starting point.

It is necessary to buffer the pH value of the aqueous solution or dispersion because the addition of the precursors (peroxygen donor and the acetyl donor) causes an increase of the pH values into the alkaline range which is not acceptable for an animal drink or animal food. This can be achieved by the addition of an organic acid.

However, the inventors have found that the reaction of the peroxygen donor and the acetyl donor is significantly affected, if the organic acid is added to the aqueous solution or dispersion together with the peroxygen donor and the acetyl donor. Unexpectedly, this inhibiting effect of the organic acid could be overcome or alleviated when it is added to the aqueous solution or dispersion only after 10 min or more, such as after 15 min or more after the preparation of the aqueous solution or dispersion comprising the peroxygen donor and the acetyl donor.

In a preferred embodiment, the organic acid is added 10 min to 4 h, such as 10 min to 3 h, such as 10 min to 2 h, after the preparation of the aqueous solution or dispersion comprising the peroxygen donor and the acetyl donor.

In a preferred embodiment, the organic acid is added 15 min to 4 h, such as 15 min to 3 h, such as 15 min to 2 h, after the preparation of the aqueous solution or dispersion comprising the peroxygen donor and the acetyl donor.

In a preferred embodiment, the organic acid is added 10 to 90 min, such as 15 to 90 min, more preferably 10 to 60 min, such as 15 to 60 min, after the preparation of the aqueous solution or dispersion comprising the peroxygen donor and the acetyl donor.

The organic acid may one organic acid or a combination of two or more organic acids. Any suitable organic acid can be used to buffer the aqueous solution or dispersion. The organic acid may be an organic compound having one or more carboxylic groups. Suitable examples for the organic acid are citric acid, organic fatty acids, sorbic acid, tartaric acid, glutaric acid, glycolic acid or any mixtures thereof, with citric acid being preferred.

The organic acid, preferably citric acid, is preferably added in an amount of 0.0020 to 0.0300 wt. %, preferably 0.0050 to 0.0250 wt. %, more preferably 0.0060 to 0.0200 wt. %, based on the weight of the aqueous solution or dispersion. It should be noted, however, that the amount of organic acid should be adjusted such that the pH of the aqueous solutions or dispersion is adjusted to a value as indicated below. The amount of the organic acid needed can therefore vary from these amounts.

For instance, the organic acid is added in such a proportion that the pH of the aqueous solution or dispersion is adjusted to a pH of 6.0 to 9.0, preferably 6.5 to 8.2, more preferably 6.5 to 7.0. The pH values refers to the values determined after the addition of the citric acid.

Accordingly, the aqueous solution of the invention preferably includes, based on the weight of the aqueous solution or dispersion,
0.0135 to 0.0315 wt. %, 0.0155 to 0.0300 wt. %, more preferably 0.0200 to 0.0250 wt. %, of the peroxygen donor, such as sodium percarbonate (SP),
0.0090 to 0.0210 wt. %, preferably 0.0100 to 0.0200 wt. %, more preferably 0.0135 to 0.0165 wt. %, of the acetyl donor, such as tetraacetylethylenediamine (TAED), and
0.0020 to 0.0300 wt. %, preferably 0.0050 to 0.0250 wt. %, more preferably 0.0060 to 0.0200 wt. %, of the organic acid such as citric acid, and
optionally a chelating agent, preferably not more than 0,050 wt. %, more preferably 0,002 to 0,040 wt. %, more preferably 0,005 to 0,020 wt. %, of a chelating agent, such as EDTA or HEDP.

The balance is typically water or water containing further additives.

The aqueous solution or dispersion achieves a suitable peracetic acid (PAA) concentration in the aqueous solution or dispersion, e.g. in the range of 3070 ppm, preferably 40-60 ppm.

The desired PAA concentration can be achieved relatively shortly after the preparation of the aqueous solution or dispersion of the invention, i.e. after addition of the organic acid. However, it can be advantageous to store or age the aqueous solution or dispersion after preparation, e.g. for at least 5 hours, preferably at least 15 hours, generally not more than 48 hours, before it is administered to the animal such as chicken.

The aqueous solution or dispersion is preferably an animal feed or animal drink, in particularly an animal drink. This refers to both the aqueous solution or dispersion to which the peroxygen donor and an acetyl donor are added and to the aqueous solution or dispersion comprising peracetic acid, i.e. the aqueous solution or dispersion comprising peracetic acid, obtained by the inventive method.

Animal drink shall be understood as any liquid given to domestic animals, especially livestock, in the course of animal husbandry. Animal drink as understood herein is usually based on water or consist of water as the sole solvent or dispersant for any actives, precursors, or additives.

The present invention is also directed to the aqueous solution or dispersion comprising peracetic acid, which is obtainable by the inventive method as described above. Of course, all general and preferred feature and embodiments described above for the inventive method also apply to the inventive aqueous solution or dispersion comprising peracetic acid.

The inventive aqueous solution or dispersion comprising peracetic acid is preferably an animal feed or animal drink, in particular an animal drink. The animal drink or animal food is preferably for poultry, such as chicken, ducks and/or turkeys, in particular chicken.

The present invention is also directed to the aqueous solution or dispersion comprising peracetic acid for use in an antimicrobial intervention to protect animals. The animal is preferably poultry, such as chicken, ducks and/or turkeys, in particular chicken.

The aqueous solution or dispersion for use in an antimicrobial intervention to protect animals according to the invention is preferably for use in the treatment or prevention of an infection of an animal, wherein the animal is preferably poultry, such as chicken, ducks and/or turkeys, in particular chicken.

Infections may comprise bacterial infections such as by (without limitation) microorganisms causing Salmonellosis, Shigellosis, Coccidiosis, E. coli infections, botulism, fowl cholera, Marek's disease, infectious bronchitis, mycoplasmosis, and infectious coryza, but not limited to these. Infections may also comprise infections by antibiotic resistant bacteria and MDROs (multi-drug resistant organisms). Infections may also comprise viral, like fowl pox, Newcastle disease and avian flu, and/or fungicidal infections.

The aqueous solution or dispersion for use in an antimicrobial intervention to protect animals according to the invention is also suitable for use to create a beneficial reduction in bacterial numbers or a beneficial alteration in bacterial spectrum in the gut microbiome in animals, wherein the animal is preferably poultry such as chicken, ducks and/or turkeys, in particular chicken.

The present invention is also directed to a kit of parts, comprising a first package and a second package, wherein the first package contains a solid mixture of a peroxygen donor, an acetyl donor and optionally a chelating agent, and the second package contains an organic acid, preferably a solid organic acid.

In a preferred embodiment of the kit of parts of the invention, the weight ratio of the peroxygen donor to the acetyl donor in the first package is from 4:1 to 1:0.5, preferably 3:1 to 1:1, more preferably 2:1 to 1:1.

In a preferred embodiment of the kit of parts of the invention, the weight ratio of the combined weight of the peroxygen donor and the acetyl donor in the first package to the weight of the organic acid in the second package is from 10:1 to 1:1, preferably 8:1 to 2:1, more preferably 6:1 to 4:1.

The first and second package can be any suitable package to store solid materials. For example, the first and second package may be a container or a pouch.

The details and preferred embodiments described above for the inventive method equally apply with respect to the inventive kit of part, in particular with respect to the peroxygen donor, the acetyl donor, the chelating agent, the organic acid and possible further additives and the proportions thereof.

The total weight of the peroxygen donor, the acetyl donor and optionally the chelating agent preferably amounts to 80 to 100% by weight, preferably 90 to 100% by weight of the content of the first package.

The total weight of the organic acid, preferably solid organic acid, preferably amounts to 80 to 100% by weight, preferably 90 to 100% by weight of the content of the second package.

The kit of parts of the invention can be suitably used to prepare the aqueous solution or dispersion of the invention, wherein the proportions of the ingredients in the first and second package are typically adjusted in a suitable manner so that the aqueous solution or dispersion of the invention can be simply prepared by adding a predetermined amount of the content of the first package to an aqueous solution or dispersion, preferably mixing the mixture obtained, storing the mixture for at least 10 min, such as for at least 15 min, and than adding an amount of the content of the second package to complete the preparation of the inventive aqueous solution or dispersion.

In an alternative embodiment of the invention, an inorganic acid instead of an organic acid is used to prepare the aqueous solution or dispersion.

According to this alternative embodiment, the invention is directed to a method for preparing an aqueous solution or dispersion comprising peracetic acid, comprising the steps of:
adding a peroxygen donor and an acetyl donor to an aqueous solution or dispersion to prepare an aqueous solution or dispersion comprising the peroxygen donor and the acetyl donor, which generate peracetic acid and hydrogen peroxide over the course of time, and
adding an inorganic acid to the aqueous solution or dispersion 10 min or later, such as 15 min or later after the preparation of the aqueous solution or dispersion comprising the peroxygen donor and the acetyl donor to buffer the pH of the aqueous solution or dispersion.

According to this alternative, the invention is also directed to a kit of parts, comprising a first package and a second package, wherein the first package contains a solid mixture of a peroxygen donor, an acetyl donor and optionally a chelating agent, and the second package contains an inorganic acid, preferably a solid inorganic acid.

These alternative embodiments of the inventive method and the inventive kit of parts differs from the respective primary embodiments of the invention only in that the organic acid is replaced by the inorganic acid. All other suitable and preferred features and embodiments described above for the primary embodiments of the invention equally apply to the alternative embodiments of the invention where an in inorganic acid is used. Hence, reference is made thereto.

The instant invention is now further illustrated in non-limiting examples.

### EXAMPLES

### Example 1

In this example, the effect of a delayed addition of organic acid (citric acid) on peracetic acid (PAA) generation was assessed.

The starting materials used are as follows:
Aqueous council water
solution: sodium percarbonate (SP)
Peroxygen donor: tetraacetylethylenediamine (TAED)
Acetyl donor: ethylenediaminetetraacetic acid (EDTA)
Chelating agent: 1-hydroxy ethylidene-1,1-diphosphonic acid

A mixture of SP, TAED and EDTA—all in solid form—are added to the stirred aqueous solution. Citric acid is added separately as a solid to the aqueous solution at varying points of time with respect to the addition of the mixture of SP, TAED and EDTA (at the same time, after 5 min, 10 min, 15 min and 30 min, respectively). For each point of time, three trials were carried out.

The pH value and the concentration of PAA in the aqueous solution was measured periodically. The PAA concentration was determined by means of a spectrophotometer. Time=0.00 hours represents the time just before the mixture of SP, TAED and EDTA is added.

### PAA Generation—Adding SP, TAED, EDTA & Citric Acid all Together

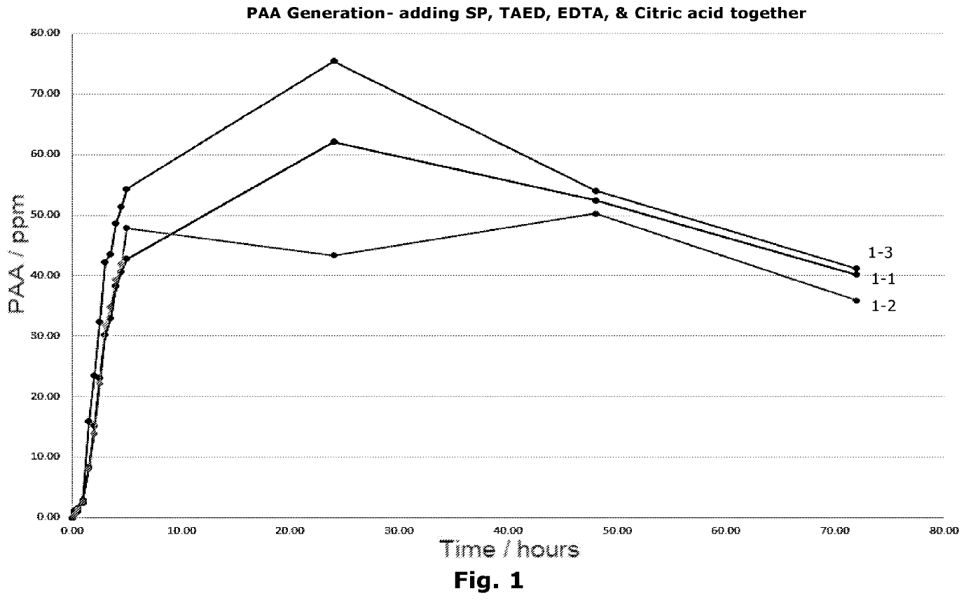

The results for the trials, where citric acid is added to the council water at the same time as the mixture of SP, TAED and EDTA are shown in the following Tables 1-1 to 1-3. A graphical representation is given in FIG. 1.

TABLE 1-1

| Time/hour | Spectrophotometer reading/abs | PAA/ppm | pH |
|---|---|---|---|
| 0.25 | 0.02 | 1.07 | 6.75 |
| 0.50 | 0.06 | 1.61 | 7.14 |
| 1.00 | 0.27 | 2.89 | 7.40 |
| 1.50 | 0.78 | 8.35 | 7.75 |
| 2.00 | 0.57 | 15.25 | 7.85 |
| 2.50 | 0.86 | 23.01 | 7.91 |
| 3.00 | 1.13 | 30.23 | 7.97 |
| 3.50 | 1.23 | 32.90 | 7.95 |
| 4.00 | 1.43 | 38.25 | 7.85 |
| 4.50 | 1.52 | 40.66 | 7.88 |
| 5.00 | 1.60 | 42.80 | 7.86 |
| 24.00 | 1.16 | 62.06 | 7.73 |
| 48.00 | 0.98 | 52.43 | 7.58 |
| 72.00 | 0.75 | 40.13 | 7.45 |

TABLE 1-2

| Time/hour | Spectrophotometer reading/abs | PAA/ppm | pH |
|---|---|---|---|
| 0.25 | 0.02 | 0.54 | 6.67 |
| 0.50 | 0.13 | 1.39 | 7.11 |
| 1.00 | 0.25 | 2.68 | 7.50 |
| 1.50 | 0.76 | 8.13 | 7.84 |
| 2.00 | 0.52 | 13.91 | 7.93 |
| 2.50 | 0.83 | 22.20 | 7.99 |
| 3.00 | 1.19 | 31.83 | 8.04 |
| 3.50 | 1.30 | 34.78 | 8.01 |
| 4.00 | 1.47 | 39.32 | 7.96 |
| 4.50 | 1.57 | 42.00 | 7.96 |
| 5.00 | 1.79 | 47.88 | 7.96 |
| 24.00 | 0.81 | 43.34 | 7.73 |
| 48.00 | 0.94 | 50.29 | 7.75 |
| 72.00 | 0.67 | 35.85 | 7.71 |

TABLE 1-3

| Time/hour | Spectrophotometer reading/abs | PAA/ppm | pH |
|---|---|---|---|
| 0.25 | 0.03 | 0.80 | 6.77 |
| 0.50 | 0.09 | 0.96 | 7.38 |
| 1.00 | 0.23 | 2.46 | 7.68 |
| 1.50 | 1.49 | 15.94 | 7.91 |
| 2.00 | 0.88 | 23.54 | 8.04 |
| 2.50 | 1.21 | 32.37 | 8.01 |
| 3.00 | 1.58 | 42.27 | 8.01 |
| 3.50 | 1.63 | 43.60 | 8.01 |
| 4.00 | 1.82 | 48.69 | 7.99 |
| 4.50 | 1.92 | 51.36 | 8.01 |
| 5.00 | 2.03 | 54.30 | 8.00 |
| 24.00 | 1.41 | 75.44 | 7.82 |
| 48.00 | 1.01 | 54.04 | 7.80 |
| 72.00 | 0.77 | 41.20 | 7.73 |

PAA Generation—Adding Citric Acid 5 Minutes after SP, TAED and EDTA

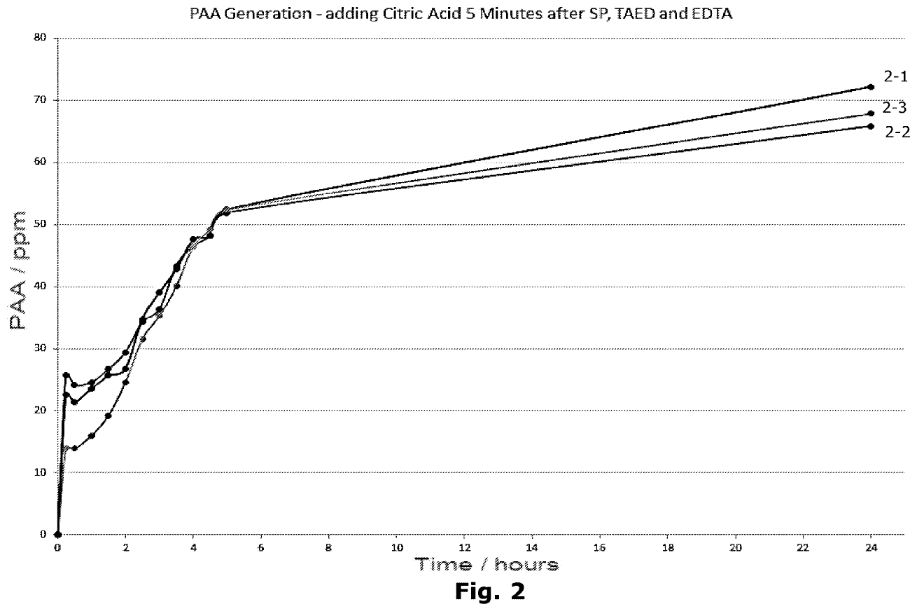

The results for the trials, where citric acid is added to the council water 5 min after the addition of the mixture of SP, TAED and EDTA are shown in the following Tables 2-1 to 2-3. A graphical representation is given in FIG. 2.

TABLE 2-1

| Time/hour | Spectrophotometer reading/abs | PAA/ppm | pH |
|---|---|---|---|
| 0.25 | 0.42 | 22.47 | 6.61 |
| 0.50 | 0.40 | 21.40 | 7.00 |
| 1.00 | 0.44 | 23.54 | 7.32 |
| 1.50 | 0.48 | 25.68 | 7.55 |
| 2.00 | 0.50 | 26.75 | 7.61 |
| 2.50 | 0.65 | 34.78 | 7.74 |
| 3.00 | 0.73 | 39.06 | 7.82 |
| 3.50 | 0.80 | 42.80 | 7.87 |
| 4.00 | 0.89 | 47.62 | 7.85 |
| 4.50 | 0.90 | 48.15 | 7.88 |
| 5.00 | 0.98 | 52.43 | 7.86 |
| 24.00 | 1.35 | 72.23 | 7.57 |

TABLE 2-2

| Time/hour | Spectrophotometer reading/abs | PAA/ppm | pH |
|---|---|---|---|
| 0.25 | 0.48 | 25.68 | 6.64 |
| 0.50 | 0.45 | 24.08 | 7.06 |
| 1.00 | 0.46 | 24.61 | 7.49 |
| 1.50 | 0.50 | 26.75 | 7.69 |
| 2.00 | 0.55 | 29.43 | 7.72 |
| 2.50 | 0.64 | 34.24 | 7.90 |
| 3.00 | 0.68 | 36.38 | 7.94 |
| 3.50 | 0.81 | 43.34 | 8.02 |
| 4.00 | 0.87 | 46.55 | 8.00 |
| 4.50 | 0.90 | 48.15 | 8.00 |
| 5.00 | 0.97 | 51.90 | 8.02 |
| 24.00 | 1.23 | 65.81 | 7.65 |

TABLE 2-3

| Time/hour | Spectrophotometer reading/abs | PAA/ppm | pH |
|---|---|---|---|
| 0.25 | 0.26 | 13.91 | 6.65 |
| 0.50 | 0.26 | 13.91 | 7.09 |
| 1.00 | 0.30 | 16.05 | 7.51 |
| 1.50 | 0.36 | 19.26 | 7.76 |
| 2.00 | 0.46 | 24.61 | 7.89 |
| 2.50 | 0.59 | 31.57 | 7.93 |
| 3.00 | 0.66 | 35.31 | 7.98 |
| 3.50 | 0.75 | 40.13 | 8.03 |
| 4.00 | 0.87 | 46.55 | 8.01 |
| 4.50 | 0.92 | 49.22 | 8.00 |
| 5.00 | 0.98 | 52.43 | 8.00 |
| 24.00 | 1.27 | 67.95 | 7.66 |

PAA Generation—Adding Citric Acid 10 Minutes after SP, TAED and EDTA

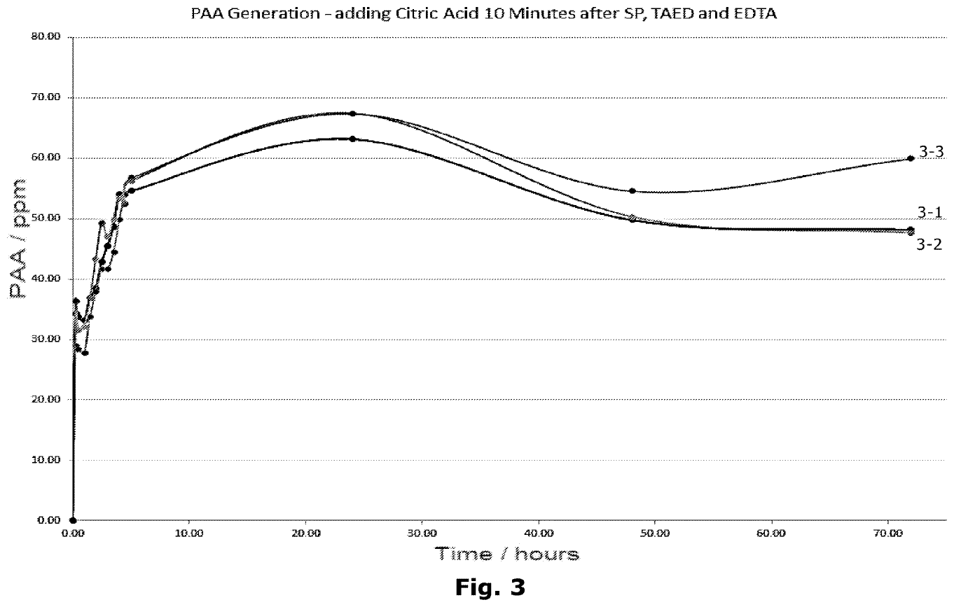

The results for the trials, where citric acid is added to the council water 10 min after the addition of the mixture of SP, TAED and EDTA are shown in the following Tables 3-1 to 3-3. A graphical representation is given in FIG. 3.

TABLE 3-1

| Time/hour | Spectrophotometer reading/abs | PAA/ppm | pH |
|---|---|---|---|
| 0.25 | 0.64 | 34.24 | 6.83 |
| 0.50 | 0.63 | 33.71 | 6.99 |
| 1.00 | 0.62 | 33.17 | 7.42 |
| 1.50 | 0.69 | 36.92 | 7.68 |
| 2.00 | 0.72 | 38.52 | 7.77 |
| 2.50 | 0.80 | 42.80 | 7.87 |
| 3.00 | 0.85 | 45.48 | 7.91 |
| 3.50 | 0.91 | 48.69 | 7.93 |
| 4.00 | 1.01 | 54.04 | 7.94 |
| 4.50 | 1.01 | 54.04 | 7.96 |
| 5.00 | 1.02 | 54.57 | 7.96 |
| 24.00 | 1.18 | 63.13 | 7.80 |
| 48.00 | 0.93 | 49.76 | 7.80 |
| 72.00 | 0.90 | 48.15 | 7.49 |

TABLE 3-2

| Time/hour | Spectrophotometer reading/abs | PAA/ppm | pH |
|---|---|---|---|
| 0.25 | 0.68 | 36.38 | 6.81 |
| 0.50 | 0.59 | 31.57 | 7.01 |
| 1.00 | 0.60 | 32.10 | 7.49 |
| 1.50 | 0.69 | 36.92 | 7.74 |
| 2.00 | 0.81 | 43.34 | 7.85 |
| 2.50 | 0.92 | 49.22 | 7.91 |
| 3.00 | 0.88 | 47.08 | 7.96 |
| 3.50 | 0.93 | 49.76 | 8.01 |
| 4.00 | 1.00 | 53.50 | 8.01 |
| 4.50 | 0.98 | 52.43 | 8.02 |
| 5.00 | 1.05 | 56.18 | 8.02 |
| 24.00 | 1.26 | 67.41 | 7.92 |
| 48.00 | 0.94 | 50.29 | 7.83 |
| 72.00 | 0.89 | 47.62 | 7.88 |

TABLE 3-3

| Time/hour | Spectrophotometer reading/abs | PAA/ppm | pH |
|---|---|---|---|
| 0.25 | 0.54 | 28.89 | 6.81 |
| 0.50 | 0.53 | 28.36 | 7.01 |
| 1.00 | 0.52 | 27.82 | 7.51 |
| 1.50 | 0.63 | 33.71 | 7.77 |
| 2.00 | 0.71 | 37.99 | 7.87 |
| 2.50 | 0.78 | 41.73 | 7.91 |
| 3.00 | 0.78 | 41.73 | 7.95 |
| 3.50 | 0.83 | 44.41 | 7.98 |
| 4.00 | 0.93 | 49.76 | 7.89 |
| 4.50 | 1.01 | 54.04 | 7.99 |
| 5.00 | 1.06 | 56.71 | 8.00 |
| 24.00 | 1.26 | 67.41 | 7.76 |
| 48.00 | 1.02 | 54.57 | 7.76 |
| 72.00 | 1.12 | 59.92 | 7.71 |

PAA Generation—Adding Citric Acid 15 Minutes after SP, TAED and EDTA

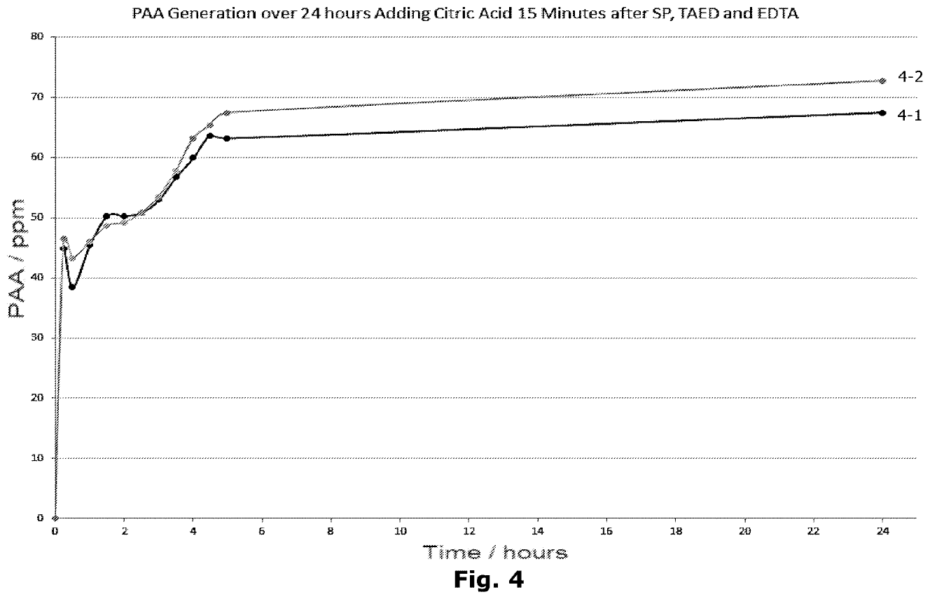

The results for the trials, where citric acid is added to the council water 15 min after the addition of the mixture of SP, TAED and EDTA are shown in the following Tables 4-1 to 4-3. A graphical representation is given in FIG. 4.

TABLE 4-1

| Time/hour | Spectrophotometer reading/abs | PAA/ppm | pH |
|---|---|---|---|
| 0.25 | 0.84 | 44.94 | 6.36 |
| 0.50 | 0.72 | 38.52 | 7.06 |
| 1.00 | 0.85 | 45.48 | 7.61 |
| 1.50 | 0.94 | 50.29 | 7.82 |
| 2.00 | 0.94 | 50.29 | 7.92 |
| 2.50 | 0.95 | 50.83 | 8.08 |
| 3.00 | 0.99 | 52.97 | 8.08 |
| 3.50 | 1.06 | 56.71 | 8.05 |
| 4.00 | 1.12 | 59.92 | 8.05 |
| 4.50 | 1.19 | 63.67 | 8.06 |
| 5.00 | 1.18 | 63.13 | 8.03 |
| 24.00 | 1.26 | 67.41 | 7.71 |

TABLE 4-2

| Time/hour | Spectrophotometer reading/abs | PAA/ppm | pH |
|---|---|---|---|
| 0.25 | 0.87 | 46.55 | 6.40 |
| 0.50 | 0.81 | 43.34 | 6.90 |
| 1.00 | 0.86 | 46.01 | 7.50 |
| 1.50 | 0.91 | 48.69 | 7.77 |
| 2.00 | 0.92 | 49.22 | 8.00 |
| 2.50 | 0.95 | 50.83 | 8.04 |
| 3.00 | 1.00 | 53.50 | 8.07 |
| 3.50 | 1.08 | 57.78 | 8.05 |
| 4.00 | 1.18 | 63.13 | 8.05 |
| 4.50 | 1.22 | 65.27 | 8.06 |
| 5.00 | 1.26 | 67.41 | 8.05 |
| 24.00 | 1.36 | 72.76 | 7.7 |

TABLE 4-3

| Time/hour | Spectrophotometer reading/abs | PAA/ppm | pH |
|---|---|---|---|
| 0.25 | 0.71 | 37.99 | 7.06 |
| 0.50 | 0.77 | 41.20 | 7.76 |
| 1.00 | 0.83 | 44.41 | 7.96 |
| 1.50 | 0.86 | 46.01 | 7.98 |
| 24.00 | 1.34 | 71.69 | 7.73 |

PAA Generation—Adding Citric Acid 30 Minutes after SP, TAED and EDTA

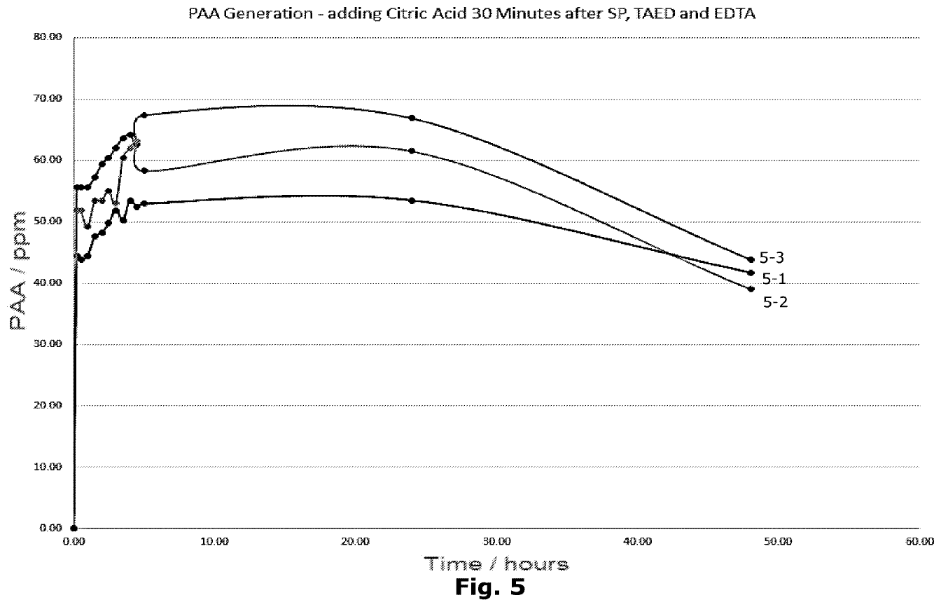

The results for the trials, where citric acid is added to the council water 30 min after the addition of the mixture of SP, TAED and EDTA are shown in the following Tables 5-1 to 5-3. A graphical representation is given in FIG. 5.

TABLE 5-1

| Time/hour | Spectrophotometer reading/abs | PAA/ppm | pH |
|---|---|---|---|
| 0.25 | 0.83 | 44.41 | 6.62 |
| 0.50 | 0.82 | 43.87 | 6.95 |
| 1.00 | 0.83 | 44.41 | 7.43 |
| 1.50 | 0.89 | 47.62 | 7.62 |
| 2.00 | 0.90 | 48.15 | 7.77 |
| 2.50 | 0.93 | 49.76 | 7.80 |
| 3.00 | 0.97 | 51.90 | 7.84 |
| 3.50 | 0.94 | 50.29 | 7.87 |
| 4.00 | 1.00 | 53.50 | 7.80 |
| 4.50 | 0.98 | 52.43 | 7.87 |
| 5.00 | 0.99 | 52.97 | 7.88 |
| 24.00 | 1.00 | 53.50 | 7.80 |
| 48.00 | 0.78 | 41.73 | 7.73 |

TABLE 5-2

| Time/hour | Spectrophotometer reading/abs | PAA/ppm | pH |
|---|---|---|---|
| 0.25 | 0.97 | 51.90 | 6.92 |
| 0.50 | 0.97 | 51.90 | 7.28 |
| 1.00 | 0.92 | 49.22 | 7.71 |
| 1.50 | 1.00 | 53.50 | 7.87 |
| 2.00 | 1.00 | 53.50 | 8.09 |
| 2.50 | 1.03 | 55.11 | 8.02 |
| 3.00 | 0.99 | 52.97 | 8.05 |
| 3.50 | 1.13 | 60.46 | 8.06 |
| 4.00 | 1.16 | 62.06 | 8.05 |
| 4.50 | 1.18 | 63.13 | 8.09 |
| 5.00 | 1.09 | 58.32 | 8.07 |
| 24.00 | 1.15 | 61.53 | 7.93 |
| 48.00 | 0.73 | 39.06 | 7.73 |

TABLE 5-3

| Time/hour | Spectrophotometer reading/abs | PAA/ppm | pH |
|---|---|---|---|
| 0.25 | 1.04 | 55.64 | 6.87 |
| 0.50 | 1.04 | 55.64 | 7.23 |
| 1.00 | 1.04 | 55.64 | 7.66 |
| 1.50 | 1.07 | 57.25 | 7.85 |
| 2.00 | 1.11 | 59.39 | 7.90 |
| 2.50 | 1.13 | 60.46 | 8.03 |
| 3.00 | 1.16 | 62.06 | 8.04 |
| 3.50 | 1.19 | 63.67 | 8.06 |
| 4.00 | 1.20 | 64.20 | 8.06 |
| 4.50 | 1.17 | 62.60 | 8.08 |
| 5.00 | 1.26 | 67.41 | 8.07 |
| 24.00 | 1.25 | 66.88 | 7.80 |
| 48.00 | 0.82 | 43.87 | 7.69 |

### Example 2

In-water peracetic acid (PAA) derived from sodium percarbonate (SP) and tetraacetylethylenediamine (TAED) precursors, could modulate poultry gut microbiota and possibly performance, functioning as a wide spectrum potential antibiotic alternative.

A total of 96 Ross 308 male day-old chickens were used during the trial duration (randomized block design with 2 rooms, 12 pens per room, 4 birds per pen and 4 pens per level of inclusion), whilst PAA treatment was administered during the last week. Six different PAA levels of inclusion were tested through in-water daily administration of SP and TAED precursors. Possible PAA effect on body weight gain (BWG), FCR (feed conversion ratio), feed/water intake and BN (bacterial number) were assessed through fitting linear mixed model (i.e. treatment as fixed effect and hierarchy of room/pen/bird when possible as random effect). Longitudinal data were used when repeated measures were taken. Total bacterial number in DNA extracted from crop, jejunum and caecal content was calculated through qPCR targeting 16S rRNA gene (V3 region). Study design and protocol were approved by SRUC Animal Welfare and Review Body (POU AE 20-2019).

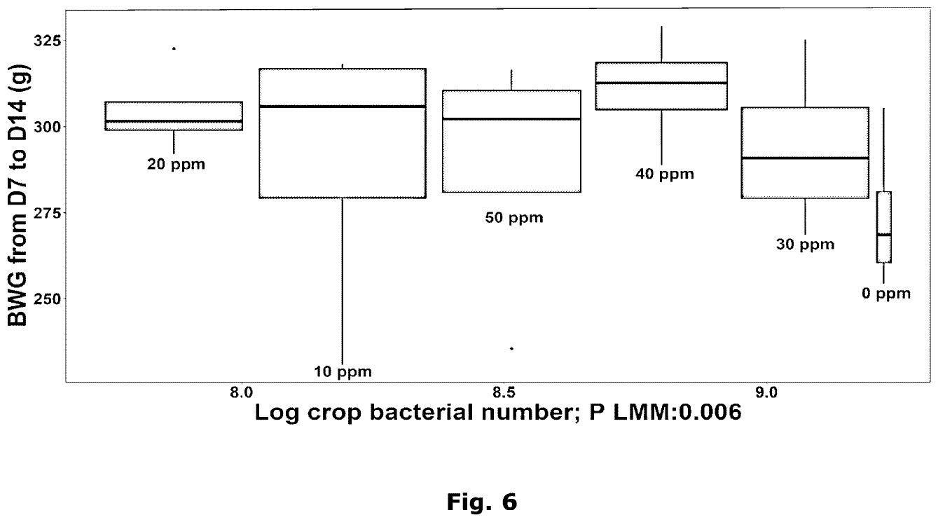

The results here presented demonstrate that PAA administered in-water for one week, through SP and TAED precursors delivery, increased BWG during treatment (P=0.05) whilst reducing BN in the crop (P=0.02) (FIG. 6), especially in one of
the experimental rooms (data not shown). Crop log 10 BN was found to be significantly correlated (P=0.006) with BWG during PM administration.

In particular, PAA administered at 40 ppm had the most evident effect on BWG from D7 to D14 (P=0.05). On the other hand, control treatment at 0 ppm was associated with the lowest BWG and highest BN (FIG. 6).

FIG. 6 shows the observed effect of PM on crop log 10 BN. The latter was significantly correlated to BWG especially when PAA was administered at 40 ppm. This could indicate a possible correlation between PAA and performance through interacting with upper-gut microbiota.

The findings presented here could therefore indicate that increasing in-water PAA level up to 50 ppm, through precursors administration, could positively affect chicken performance through interacting with upper-gut bacterial population opening the way for further studies investigating PAA administration in-water or in-feed. PAA administration was not correlated with bird mortality (0⁰/0) or side effect in general.

Whilst no adverse effects or mortality were correlated with PAA in-water administration, the concentration of total number of bacteria in crop content appears to be reduced by higher dosage. This benefit correlated with an increase BWG indicating that lowering total crop bacteria, here achieved through PAA administration, could enhance performance.

### Example 3

In this study, very high quantities of ingredients were used so that high concentrations of PM are generated. Such high PAA concentrations are not suitable for practical applications to animals. The aim of these tests is only to study the influence of different organic acids in the reaction.

An aqueous solution was prepared by adding the following ingredients in water

| Ingredient | Amount/°/0 | Amount/g |
|---|---|---|
| SP | 53.19 | 30.23 |
| TAED | 26.96 | 15.32 |
| Citric acid | 19.01 | 10.80 |
| HEDP | 0.84 | 0.48 |

The PAA concentration in the aqueous solution was determined within a span of 2 hours after addition of the ingredients.

The test was repeated except that citric acid was replaced by the following acids: tartaric acid, sorbic acid, glutaric acid, glycolic acid, and a combination of citric and tartaric acid, respectively.

In all tests, the PAA generation determined over the time is similar (in the range of about 6000 to 9000 ppm). It is concluded that the influence of the specific type of organic acid is negligible.

### Example 4

In this study, the impact of the supply of the aqueous solution or dispersion of the invention to the animal drink for a group of chickens in a shed was tested.

Aqueous solutions used were prepared with a first powder consisting of a mixture of SP, TAED and EDTA and a second powder which is citric acid. The aqueous solutions obtained had the following composition:

| Ingredient | % by weight |
|---|---|
| Water | 99.9445 |
| TAED | 0.015 |
| SP | 0.0225 |
| EDTA | 0.01 |
| Citric Acid | 0.008 |

The aqueous solution is prepared and supplied as the animal drink for the chickens in the shed according to the following procedure:
Step 1: add the first powder (SP, TAED and EDTA) to the predetermined volume of water.
Step 2: mix for 15 minutes.
Step 3: add corresponding quantity of second powder (citric acid).
Step 4: mix for further 15 min
Step 5: leave for 24 hours.
Step 6: link the completed mix to the water line for the shed and dispense through filter and dosing equipment to deliver the desired concentration of PAA, 30-70 ppm, throughout the shed. Concentration checked at various points, nipple drinkers and pipe ends.

The amount of citric acid was adjusted to obtain a pH between 6 and 9 in the aqueous solution. The time span of the study was from day 1 to day 39. Different amounts of aqueous solutions were prepared for each day of supply based on the amount of water consumed (as the chickens grow they require more water).

At days 1 to 5 conventional animal drink was supplied to the water line for the shed, whereas at days 6 to 39 the inventive aqueous solution as prepared above was supplied to the water line for the shed in quantities as shown in the following table.

| Animal drink | Total water/ [symbol] | Day | Dose/% [symbol] |
|---|---|---|---|
| conventional | 460 | 1 | 0 |
| conventional | 1215 | 2 | 0 |
| conventional | 1640 | 3 | 0 |
| conventional | 1590 | 4 | 0 |
| conventional | 2030 | 5 | 0 |
| inventive | 2220 | 6 | 4 |
| inventive | 2575 | 7 | 4 |
| inventive | 2895 | 8 | 3 |
| inventive | 3260 | 9 | 2.5 |
| inventive | 3710 | 10 | 2 |
| inventive | 4200 | 11 | 2 |
| inventive | 4425 | 12 | 2 |
| inventive | 4835 | 13 | 2 |
| inventive | 5220 | 14 | 1.5 |
| inventive | 5760 | 15 | 1.5 |
| inventive | 6585 | 16 | 1.5 |
| inventive | 6625 | 17 | 1.5 |
| inventive | 7135 | 18 | 1.5 |
| inventive | 7660 | 19 | 1 |
| inventive | 7490 | 20 | 1 |
| inventive | 7425 | 21 | 1 |
| inventive | 8140 | 22 | 1 |
| inventive | 8065 | 23 | 1 |
| inventive | 8770 | 24 | 1 |
| inventive | 8720 | 25 | 1 |
| inventive | 8675 | 26 | 1 |
| inventive | 8345 | 27 | 1 |
| inventive | 6130 | 28 | 1.5 |
| inventive | 6180 | 29 | 1.5 |
| inventive | 6555 | 30 | 1.5 |
| inventive | 6625 | 31 | 1.5 |
| inventive | 6580 | 32 | 1.5 |
| inventive | 6165 | 33 | 1.5 |
| inventive | 6015 | 34 | 1.5 |
| inventive | 6330 | 35 | 1.5 |
| inventive | 6980 | 36 | 1.5 |
| inventive | 7255 | 37 | 1.5 |
| inventive | 7485 | 38 | 1 |
| inventive | 7460 | 39 | 1 |

| | |
|---|---|
| 219430 | I Total for 39 |

[symbol]

indicates data missing or illegible when filed

### Example 5

In this trial 34,540 birds were housed into shed 2. An aqueous solution of the invention was prepared essentially as in Example 4. Each aqueous solution was prepared the day before and fed through the Dosatron in the water lines at varying percentages (between 1-5%). Samples were taken from the lines and analyzed for PAA content and it was determined that between 30-50 ppm PAA was generated. Vitamins were fed for the first 5 days. PAA was fed from day 6 to day 41.

During the test, dead chickens, leg cull and other culls were determined for each day, and on days 7, 14, 21 and 28 the average weight of the chicken were determined. The results are given in the following table.

### Trial

Shed 2 data—Nov. 10, 2022
Batch
77
34540 Chickens

| | | | Leg | Other | Total | Cumulative | Total | Mean | |
|---|---|---|---|---|---|---|---|---|---|
| Day | Date | Dead | Culls | Culls | Daily | Total | water/L | weight | |
| 1 | 12 Oct. 2022 | 114 | — | — | 114 | 114 | 510 | | |
| 2 | 13 Oct. 2020 | 68 | — | 28 | 96 | 210 | 1425 | | |
| 3 | 14 Oct. 2020 | 46 | — | 23 | 69 | 279 | 1760 | | |
| 4 | 15 Oct. 2020 | 33 | — | 15 | 48 | 327 | 1885 | | |
| 5 | 16 Oct. 2020 | 36 | — | 12 | 48 | 375 | 2155 | | |
| 6 | 17 Oct. 2020 | 12 | | 6 | 18 | 393 | 2150 | | |
| 7 | 18 Oct. 2020 | 15 | 1 | 4 | 20 | 413 | 2425 | 197 | g |
| 8 | 19 Oct. 2020 | 8 | 2 | 5 | 15 | 428 | 2555 | | |
| 9 | 20 Oct. 2020 | 14 | 2 | 5 | 21 | 449 | 2520 | | |
| 10 | 21 Oct. 2020 | 16 | 2 | 4 | 22 | 471 | 3030 | | |
| 11 | 22 Oct. 2020 | 8 | — | 3 | 11 | 482 | 3280 | | |
| 12 | 23 Oct. 2020 | 9 | 2 | 3 | 14 | 496 | 3770 | | |
| 13 | 24 Oct. 2020 | 13 | 1 | 6 | 20 | 516 | 4070 | | |
| 14 | 25 Oct. 2020 | 7 | — | 2 | 9 | 525 | 4550 | 518 | g |
| 15 | 26 Oct. 2020 | 8 | — | 1 | 9 | 534 | 4590 | | |
| 16 | 27 Oct. 2020 | 10 | 1 | 2 | 13 | 547 | 5660 | | |
| 17 | 28 Oct. 2020 | 4 | 1 | — | 5 | 552 | 6070 | | |
| 18 | 29 Oct. 2020 | 5 | — | — | 5 | 557 | 6920 | | |
| 19 | 30 Oct. 2020 | 5 | — | 2 | 7 | 564 | 7210 | | |
| 20 | 31 Oct. 2020 | 6 | — | 5 | 11 | 575 | 6835 | | |
| 21 | 1 Nov. 2020 | 10 | . . . — | 2 | 2 | 587 | 7305 | 1150 | g |
| 22 | 2 Nov. 2020 | 19 | — | 5 | 24 | 611 | 7065 | | |
| 23 | 3 Nov. 2020 | 9 | — | 4 | 13 | 624 | 8130 | | |
| 24 | 4 Nov. 2020 | 20 | | 2 | 22 | 646 | 7440 | | |
| 25 | 5 Nov. 2020 | 20 | — | 3 | 23 | 669 | 8125 | | |
| 26 | 6 Nov. 2020 | 13 | 2 | — | 15 | 684 | 8830 | | |
| 27 | 7 Nov. 2020 | 10 | — | 4 | 14 | 698 | 8910 | | |
| 28 | 8 Nov. 2020 | 11 | — | 2 | 13 | 711 | 4455 | 1450 | g |
| 29 | 9 Nov. 2020 | 16 | — | 3 | 19 | 730 | 9300 | | |
| 30 | 10 Nov. 2020 | 7 | — | 1 | 00 | 738 | 7230 | | |
| 31 | 11 Nov. 2020 | 5 | — | — | 5 | 743 | 7460 | | |
| 32 | 12 Nov. 2020 | 5 | 1 | — | 0 | 749 | 7455 | | |
| 33 | 13 Nov. 2020 | 5 | — | 1 | 0 | 755 | 7290 | | |
| 34 | 14 Nov. 2020 | 5 | — | — | 5 | 760 | 7395 | | |
| 35 | 15 Nov. 2020 | 12 | — | 4 | 16 | 776 | 7288 | | |
| 36 | 16 Nov. 2020 | 11 | — | 4 | 15 | 791 | 7385 | | |
| 37 | 17 Nov. 2020 | 6 | | 2 | 8 | 799 | 5880 | | |
| 38 | 18 Nov. 2020 | 6 | | 4 | 10 | 809 | 6110 | | |
| 39 | 19 Nov. 2020 | 4 | — | — | 4 | 813 | 6285 | | |
| 40 | 20 Nov. 2020 | 6 | — | 5 | 11 | 824 | 6890 | | |
| 41 | 21 Nov. 2020 | 5 | 3 | | 8 | 832 | 7030 | | |

| | |
|---|---|
| 231628 | L |
| 0.6533935 | L per |

The results of the study with a treatment at target concentrations of about 50 ppm PAA derived from the aqueous solution was compared with two control groups (sheds 3 and 4) and the results of the inventive treatment can be summarized as follows:
No negative impact on mortality or morbidity.
No observations of unusual/changed behaviors.
Controls were given same vitamins and Huwa-San was continued in shed 3 and 4. Huwa-San TR-50 was discontinued from day 1 of the trial in shed 2 (Huwa-San TR-50 is a 50% hydrogen peroxide solution used to clean the water lines).
Gumboro and pneumonia vaccinations were not given in shed 2.
Litter quality was observed to be improved in shed 2.
Birds weights were in line with expected performance.
Costs of raw materials were calculated at 1.23p per bird for 35 days (ignoring 800 dead birds becomes 1.26p if these are taken into account).
6.72 L of water per bird over 41 days was consumed (231628 L of water in total for 35000 birds over 41 days).
Cost saving of vaccinations for Gumboro and pneumonia were 1.25p per bird.
Shed 3 & 4 needed to have antibiotics. Extra cost for antibiotics for shed 3 & 4 were 1.5p per bird.
Birds in all 3 sheds were the same batch and would be expected to have the same gut microbiome.
Shed 3 & 4 tested positive for Salmonella at around day 21.
Shed 2 birds never tested positive for Salmonella.
Veterinary examination found birds to be in good condition and superior to bird samples in other sheds.

1-22. (canceled)

23. A method for preparing an aqueous solution or dispersion comprising peracetic acid, the method comprising the steps of:
adding a peroxygen donor and an acetyl donor to an aqueous solution or dispersion to prepare an aqueous solution or dispersion comprising the peroxygen donor and the acetyl donor, which generate peracetic acid and hydrogen peroxide over time, and
adding an organic acid to the aqueous solution or dispersion 10 minutes or later after preparation of the aqueous solution or dispersion comprising the peroxygen donor and the acetyl donor to buffer a pH of the aqueous solution or dispersion.

24. The method according to claim 23, wherein the organic acid is selected from citric acid, organic fatty acids, sorbic acid, tartaric acid, glutaric acid, glycolic acid, or any mixtures thereof; or wherein the peroxygen donor is selected from sodium perborate, ammonium perborate, sodium percarbonate, potassium percarbonate, ammonium percarbonate, sodium perphosphate, ammonium persulphate, urea peroxide, peresters, superoxides, dioxygenyl, ozones, hydrogen peroxide, lithium peroxide, barium peroxide, di-tert-butyl peroxide, ammonium peroxydisulphate and potassium peroxymonosulphate or any mixture thereof; or wherein the acetyl donor is selected from tetraacetylethylenediamine (TAED), methyl cellulose encapsulated TAED, acetyl salicylic acid, diacetyl dioxohexahydratriazine (DADHT), tetraacetyl glycoluril, acetyl urea, di-acetyl urea, tri-acetyl urea, pentaacetyl glucose (PAG), tetraacetyl glycoluril (TAGU), acetyl phosphate, acetyl imidazole, acetyl COA, acetic anhydride, compounds containing a hemiacetal group, acetic acid, di-, acetylmorphine, pyruvate, acetyl chloride, acetyl-caprolactam and N′N′-diacetyl-N′N′-dimethyl urea or any mixture thereof.

25. The method according to claim 23, wherein the organic acid is added in such a proportion that the pH of the aqueous solution or dispersion is adjusted to a pH of 6.0 to 9.0.

26. The method according to claim 23, wherein the organic acid is added 10 to 90 minutes after preparation of the aqueous solution or dispersion comprising the peroxygen donor and the acetyl donor.

27. The method according to claim 23, wherein the aqueous solution or dispersion to which the peroxygen donor and the acetyl donor is added is water.

28. The method according to claim 23, wherein the aqueous solution or dispersion to which the peroxygen donor and the acetyl donor is added has a pH value in a range of 5.0 to 9.5.

29. The method according to claim 23, wherein the peroxygen donor and the acetyl donor are added to the aqueous solution or dispersion such that a weight ratio of the peroxygen donor to the acetyl donor is from 4:1 to 1:0.5.

30. The method according to claim 23, wherein, based on a weight of the aqueous solution or dispersion, the peroxygen donor is added in an amount in a range of 0.0135 to 0.0315 wt. %, and/or the acetyl donor is added in an amount in a range of 0.0090 to 0.0210 wt. %, and/or the organic acid is added in an amount of 0.0020 to 0.0300 wt. %.

31. The method according to claim 23, wherein the aqueous solution or dispersion comprising the peroxygen donor and the acetyl donor further comprise a chelating agent, wherein an amount of the chelating agent is not more than 0.050 wt. % based on a weight of the aqueous solution or dispersion.

32. The method according to claim 23, wherein the peroxygen donor and the acetyl donor are added as a solid mixture to the aqueous solution or dispersion or, when a chelating agent is included, the peroxygen donor, the acetyl donor and the chelating agent are added as the solid mixture to the aqueous solution or dispersion.

33. The method according to claim 23, wherein the aqueous solution or dispersion comprising the peroxygen donor and the acetyl donor is stirred.

34. The method according to claim 23, wherein the aqueous solution or dispersion is an animal feed or animal drink.

35. An aqueous solution or dispersion comprising peracetic acid, obtainable by the method according to claim 23, wherein the aqueous solution or dispersion comprising peracetic acid is an animal feed or animal drink.

36. The aqueous solution or dispersion according to claim 37 for use in an antimicrobial intervention to protect animals.

37. The aqueous solution or dispersion for use in the antimicrobial intervention to protect animals according to claim 38, for use in treatment or prevention of an infection or for use to create a beneficial reduction in bacterial numbers or a beneficial alteration in bacterial spectrum in a gut microbiome in animals.

38. A kit of parts, comprising a first package and a second package, wherein the first package contains a solid mixture of a peroxygen donor, an acetyl donor and optionally a chelating agent, and the second package contains an organic acid.

39. The kit of parts according to claim 40, wherein a weight ratio of the peroxygen donor to the acetyl donor in the first package is from 4:1 to 1:0.5.

40. The kit of parts according to claim 40, wherein a weight ratio of a combined weight of the peroxygen donor and the acetyl donor in the first package to a weight of the organic acid in the second package is from 10:1 to 1:1.

41. The kit of parts according to claim 40, wherein the first package and the second package are independently a container or a pouch.

42. The kit of parts according to claim 40, wherein the peroxygen donor is selected from sodium perborate, ammonium perborate, sodium percarbonate, potassium percarbonate, ammonium percarbonate, sodium perphosphate, ammonium persulphate, urea peroxide, peresters, superoxides, dioxygenyl, ozones, hydrogen peroxide, lithium peroxide, barium peroxide, di-tert-butyl peroxide, ammonium peroxydisulphate and potassium peroxymonosulphate or any mixture thereof; the acetyl donor is selected from tetraacetylethylenediamine (TAED), methyl cellulose encapsulated TAED, acetyl salicylic acid, diacetyl dioxohexahydratriazine (DADHT), tetraacetyl glycoluril, acetyl urea, di-acetyl urea, tri-acetyl urea, pentaacetyl glucose (PAG), tetraacetyl glycoluril (TAGU), acetyl phosphate, acetyl imidazole, acetyl COA, acetic anhydride, compounds containing a hemiacetal group, acetic acid, di-, acetylmorphine, pyruvate, acetyl chloride, acetyl-caprolactam and N′N′-diacetyl-N′N′-dimethyl urea or any mixture thereof; and the organic acid is selected from citric acid, organic fatty acids, sorbic acid, tartaric acid, glutaric acid, glycolic acid, or any mixtures thereof.
